(19) United States
(12) Patent Application Publication
(10) Pub. No.: US 2025/0213232 A1
(43) Pub. Date: Jul. 3, 2025

(54) SYSTEMS AND METHODS FOR CALCULATING VOLUMES OF EXCRETED URINE AND STOOL

(71) Applicant: DUKE UNIVERSITY, Durham, NC (US)

(72) Inventors: Lawrence DAVID, Durham, NC (US); Stephen EMBREE, Durham, NC (US); Benjamin NEUBERT, Durham, NC (US)

(21) Appl. No.: 18/867,588

(22) PCT Filed: May 19, 2023
(86) PCT No.: PCT/US2023/022911

§ 371 Date: Nov. 20, 2024

Related U.S. Application Data
(60) Provisional application No. 63/344,302, filed on May 20, 2022.

(51) Int. Cl. A61B 10/00; A61B 5/00; A61B 5/20; G01F 23/20; G01F 23/80; G01N 33/493

(52) U.S. Cl. CPC A61B 10/0038; A61B 5/208; A61B 5/6891; A61B 10/007; G01F 23/20; G01F 23/80; G01N 33/493

(57) ABSTRACT

An excretion collection and evaluation system including at least one waste receptacle including a waste collection area. and a sensor configured to be coupled to a toilet, selectively coupled to the at least one waste receptable, and configured to generate a signal indicative of a quantity of waste deposited in the waste collection area.

### RELATED APPLICATIONS

This application claims priority to and the benefit of U.S. Provisional Patent Application No. 63/344,302 filed May 20, 2022, which is incorporated herein by reference in its entirety for all purposes.

### FIELD

This present disclosure provides systems and methods for evaluating excretions from a subject. In some aspects, provided herein are systems that can be used in conjunction with commercially available toilets to evaluate and quantify fecal and/or urine output from a subject.

### BACKGROUND

Hospital treatment of diseases related to the heart, lungs, and blood often requires tracking patient urine and stool output. For patients who can walk, waste output monitoring is currently a manual process. Patients are instructed to urinate and defecate into plastic collection hats that are placed under toilet seats. When these hats are filled or at the end of shifts, nurses will visually inspect hat contents and record volumes according to marked gradations on the hat walls. If both urine and stool are present in the same hat, volume adjustments must be estimated. Afterwards, nurses will empty hat contents into the toilet, rinse and clean them, and return them to the toilet for patient reuse. The manual nature of patient and nurse handling of collection hats can lead to substantial error in patient fluid balances, which has multiple adverse consequences including increased mortality, prolonged length of hospital stay, higher ventilator use, and increased hospital expenses. Beyond patient health, existing practices for handling hats also expose nurses to active chemotherapeutics excreted in patient waste. Moreover, when diarrhea and urine are mixed in the same collection hat, it is difficult to separately estimate volumes of each. Thus, there is an ongoing need for advances in how urine and stool are tracked, in order to both improve the care of patients and protect the health of nurses who care for them.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one implementation, the present disclosure provides an excretion collection and evaluation system that includes at least one waste receptacle including a waste collection area, and a sensor configured to be coupled to a toilet, selectively coupled to the at least one waste receptable, and configured to generate a signal indicative of a quantity of waste deposited in the waste collection area.

In some implementations, the sensor includes at least one load cell that interfaces with the at least one waste receptacle.

In some implementations, the at least one waste receptacle includes an overhanging region selectively coupled to the sensor.

In some implementations, the overhanging region includes a receptacle locking feature configured to constrain movement of the at least one waste receptacle relative to the sensor.

In some implementations, the sensor includes a sensor locking feature configured to engage the at least one waste receptacle to constrain movement of the at least one waste receptacle relative to the sensor.

In some implementations, the receptacle locking feature includes an aperture, and the sensor locking feature includes a protrusion sized to engage the aperture.

In some implementations, the at least one waste receptacle includes a urine receptacle and a stool receptacle. In some implementations, the urine receptacle and the stool receptacle are independently coupled to the sensor.

In some implementations, the at least one waste receptacle includes a urine receptacle and a stool receptacle. In some implementations, the urine receptacle and the stool receptacle are combined in a single sample collection container.

In some implementations, the at least one waste receptacle includes a urine receptacle.

In some implementations, the at least one waste receptacle includes a stool receptacle.

In some implementations, the at least one waste receptacle includes a drain.

In some implementations, the at least one waste receptacle defines slopes to direct a flow of fluid towards the drain.

In some implementations, the excretion collection and evaluation system further includes a separator configured to inhibit contact between a toilet seat, the sensor, or the at least one waste receptacle. In some implementations, the at least one waste receptacle includes a cutout sized to receive the separator, and wherein the separator is secured to the toilet seat.

In some implementations, the excretion collection and evaluation system further includes a controller that receives the signal from the sensor and evaluates a weight or volume of excretions based on the signal.

In some implementations, the controller is configured to transmit data to an electronic medical station.

In another implementation, an excretion collection and evaluation system includes a toilet seat configured to be hingedly coupled to a toilet and including a separator extending from a bottom surface of the toilet seat, and a quantity measurement sensor coupled to the toilet seat and positioned so that the separator inhibits contact between the toilet seat and the weight measurement sensor.

In some implementations, the quantity measurement sensor includes a battery powered load cell.

In some implementations, the quantity measurement sensor is configured to be directly coupled with the toilet and the toilet seat includes a toilet seat hinge configured to couple to the toilet seat to the quantity measurement sensor.

In another implementation, a waste receptacle for use with an excretion collection and evaluation system includes a waste collection area, a support flange extending from the waste collection area and including a receptacle locking feature, and a drain positioned within the waste collection area configured to allow excretions to exit the waste collection area during use. The support flange is configured to: suspend the waste collection area within a toilet bowl, constrain movement of the waste receptacle relative to the excretion collection and evaluation system, and engage a quantity measurement sensor of the excretion collection and evaluation system.

In some implementations, the waste collection area includes a urine receptacle and a separate stool receptacle.

### BRIEF DESCRIPTION OF THE DRAWINGS

The accompanying figures are provided by way of illustration and not by way of limitation.

FIGS. 1A-1C show representative illustrations of an exemplary system for evaluating excretions from a subject, according to one embodiment of the present disclosure. FIG. 1A is a perspective view showing the system with the toilet seat raised and without the sample collection containers FIG. 1B is a perspective view highlighting placement of the sample collection containers upon the scale. FIG. 1C is a perspective view highlighting how the multiple components of the device can pivot around the hinge. FIG. ID is a perspective view highlighting the proper positioning of the separators on the underside surface of the toilet seat such that the load cells and the overhanging regions of the sample collection container are not contacted by the separators when the toilet seat is lowered. FIG. 1E is a representative illustration showing a zoomed in view of the hinge.

FIGS. 2A-2B show representative illustrations of an exemplary sample collection container for use in the systems and methods provided herein, according to one embodiment of the present disclosure. FIG. 2A is a perspective view of the exemplary collection container. FIG. 2B is a top view of the exemplary sample collection container.

FIGS. 3A-3D show representative photographs of an exemplary system for evaluating urine excretions from a subject, according to one embodiment of the present disclosure, located in a hospital bathroom. FIG. 3A is a photograph showing an exemplary system absent a sample collection container. FIG. 3B is a photograph highlighting placement of the sample collection container. FIG. 3C is a photograph showing a different view of the system shown in FIG. 3B. FIG. 3D is a photograph showing the system with the toilet seat lowered. The photograph highlights proper placement of the separators on the underside surface of the toilet seat.

FIGS. 4A-4D show photographs of an exemplary system for evaluating excretions from a subject in accordance with one embodiment of the present disclosure. FIG. 4A shows a photograph of an exemplary scale in accordance with one embodiment of the present disclosure. FIG. 4B shows a photograph of an exemplary system including waste receptacles. FIG. 4C is a photograph showing the system including a conventional, off-the-shelf toilet seat for patient comfort. FIG. 4D is a photograph showing the double-hinged design of the system.

FIGS. 5A-5C show an exemplary system operation schematic in accordance with one embodiment of the present disclosure. FIG. 5A shows a schematic of how the system processes patient waste. Direct weight measurements are sufficient for stool mass estimates. Rates of changing weights of the sample collection container over time can be used to estimate urine volume and diarrheal incidence. FIG. 5 shows a characteristic curve of urination collection container weight as urine accumulates and drains out. FIG. 5C shows mathematical model relating volume of liquid in hat at a given time (V_{liquid}(t); Eq. 1), weight of hat over time (W_{liquid}(t); Eq. 2); change in liquid volume over time (dVₒᵤₜ/dt; Eq. 3); and, total volume of liquid that enters and leaves hat (Vₒᵤₜ; Eq. 4). Measured weights over time (e.g., curve shown in (FIG. 5B)) are used to solve Eq. 4. Constants in Eq. 4 are estimated via calibration using known input volumes.

FIGS. 6A-6B show experiments demonstrating accuracy of the system and method. FIG. 6A is a graph showing simulated urine outputs plotted against measured values (n=25 simulations). Shown also are mean absolute errors (MAE) and mean absolute percent errors (MAPE). FIG. 6B is a graph showing error during use by healthy volunteers (n=22 uses).

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “container”, “collection container”, or “sample collection container” are used in the broadest sense and refers to any suitable body used to collect a urine or a stool sample from a subject. In some embodiments, a sample collection container comprises a urine receptacle for collecting urine from a subject. In some embodiments, a sample collection container comprises a stool receptacle for collecting stool from a subject. In some embodiments, a sample collection container comprises a urine receptacle for collecting urine from a subject and a stool receptacle for collecting stool from the subject.

As used herein, “evaluating excretions” is used in the broadest sense and refers to any method of observing, identifying, or measuring urine and/or fecal excretions from a subject. In some embodiments, “evaluating” refers to measuring urine excretions and/or fecal excretions from a subject. Measuring such excretions can refer to measuring the weight and/or the volume of the excretions. For example, “evaluating” urine excretions can refer to measuring the weight and/or volume of urine excreted by the subject in a given excretion event or over the course of a given window of time. As another example, “evaluating” fecal excretions can refer to measuring the weight and/or volume of stool excreted by the subject in a excretion event or over the course of a given window of time. In some embodiments, “evaluating” fecal excretions can refer to determining the amount (e.g., by weight or by volume) of solid stool vs. diarrhea in a given excretion event or over the course of a given window of time

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

2. Systems and Methods for Evaluating Excretions from a Subject

Embodiments of the present disclosure include systems and methods of use thereof for evaluating excretions from a subject, including urine and/or stool excretions. In some embodiments, the systems and methods described herein find use in methods of evaluating excretions from the subject in the context of monitoring various disease, conditions, or responses to the subject to one or more therapeutic agents. The present disclosure relates generally to systems that can be used to collect information related to a subject's elimination events (e.g., diagnostic information) as well as to improve efficiency and convenience of toilet usage, both at home and in the setting of a care facility (e.g., hospital, clinic, nursing home, and the like). The systems disclosed herein are particularly useful in a therapeutic setting, such as a hospital or clinic, in which excretions from a subject are observed for a variety of treatment purposes. For example, information related to urine and/or fecal excretions from a subject can be used to assist in making a determination regarding a subject's health or disease state, making a determination regarding a subject's recovery from medical treatment or procedure, and/or making a determination regarding a subject's response to a particular therapeutic agent. These and other data can be collected in an automatic manner by the disclosed systems and thus avoid the need for manual evaluation of urine or fecal output from a subject. Additionally, the systems described herein provide a cost-effective method for evaluating excretions from a subject and can be used in conjunction with any standard toilet, without requiring changes to standard plumbing or the toilet itself.

In some aspects, the systems for evaluating excretions from a subject described herein can be used to measure the contents of waste receptacles, such as waste collection “hats” commonly found in hospitals. Waste receptacles are also referred to herein as “waste collection containers” and “sample collection containers”. Waste receptacles are commonly used in the course of patient care for tracking patient fluid status and for determining the severity of diseases related to diarrheal output. In the systems provided herein, the container(s) used to collect excretions from the subject are distinct from the component of the system that measures the urine and/or fecal output from the subject. In other words, the waste receptacle itself is not equipped for automated measurement of excretions from the subject. Accordingly, the waste receptacle can be obtained in a cost-effective and simple manner without the need to equip commercially available containers for automated measurements, or to manufacture custom containers containing expensive equipment, such as scales, sensors, detectors, and the like.

In some aspects, provided herein is a system for evaluating excretions from a subject. In some embodiments, the system comprises a quantity measurement sensor. In some embodiments, the quantity measurement sensor is configured to be coupled to a toilet. In some embodiments, the quantity measurement sensor is configured for hinged attachment to a toilet. In some embodiments, the quantity measurement sensor is configured to be positioned (e.g. attached via a hinge) above the rim of the toilet and below the toilet seat. In some embodiments, the quantity measurement sensor is configured to be selectively coupled to at least one waste receptable, as described in more detail below. In some embodiments, the quantity measurement sensor is configured to generate a signal indicative of a quantity of waste deposited in the waste collection area.

In some implementations, the quantity measurement sensor may be any type of weight sensor. In some implementations, the quantity measurement sensor includes an optical sensor (e.g., an IR sensor), a hall effect strain gauge, laser sensors, a volume sensor, an ultrasound sensor, a resistance sensor, a capacitance sensor, or any other type of sensor that produces a signal indicative of a quantity of excretions deposited in the waste receptacle. In some embodiments, the quantity measurement sensor comprises a scale. In some embodiments, the scale is configured for hinged attachment to a toilet. In some embodiments, the scale can be positioned (e.g. attached via a hinge) above the rim of the toilet and below the toilet seat. In some embodiments, the scale comprises a quantity measurement sensor in the form of one or more load cells. In some embodiments, the quantity measurement sensor (e.g. the scale) comprises one or more load cells. The term “load cell” refers to the portion of the scale that measures weight. In some embodiments, the load cells are configured to interface with a waste receptacle. In some embodiments, the one or more load cells are positioned on the scale such that they come into contact with a receptacle support flange in the form of an overhanging region of at least one waste receptacle to measure waste (e.g. urine and/or stool) output from a subject. For example, in some embodiments the scale comprises one or more load cells positioned spatially in locations on the scale that correspond to locations of an overhanging region of a waste receptacle (e.g., a lip of the container, which overhangs onto a suitable solid surface to prevent the collection container from falling into the toilet during use). Accordingly, the overhanging region of the waste receptacle comes into contact with the load cell and thus a force placed on the waste receptacle (e.g., weight in the receptacle, due to urine and/or stool) conveys force onto the load cell, thus facilitating quantification of excretions from the subject.

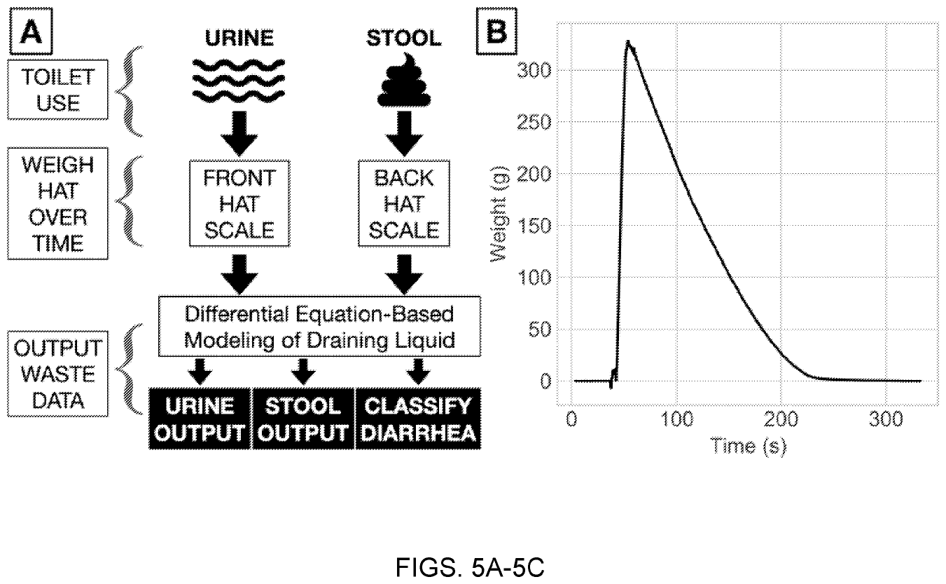

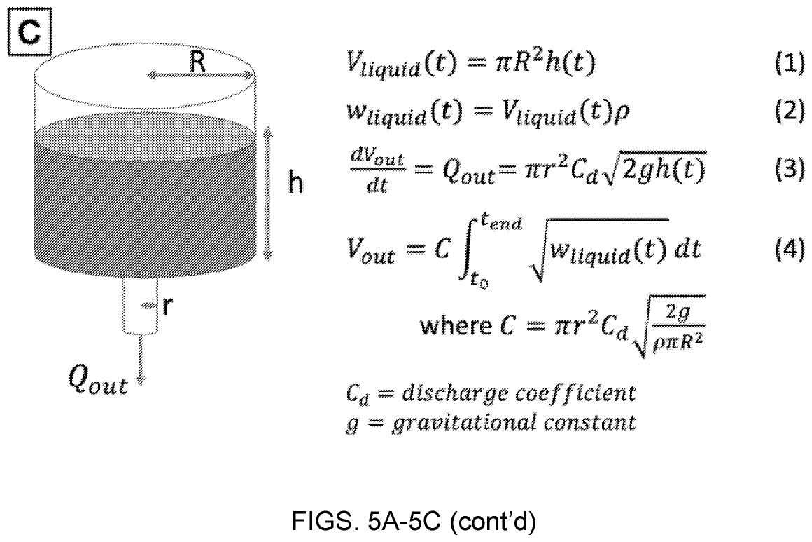

The one or more load cells may be any suitable size and any suitable shape to facilitate placement within the scale and evaluation of excretions from the subject. In some embodiments, the scale comprises at least one urine load cell. The at least one urine load cell quantifies (e.g., weighs) urine excretions from a subject. In some embodiments, the scale comprises two urine load cells. For example, in some embodiments, the scale comprises a urine load cell on the left side of the scale and a urine load cell on the right side of the scale. In some embodiments, the scale comprises at least one stool load cell. The at least one stool load cell quantifies stool excretions from a subject. In some embodiments, the scale comprises two stool load cells. In some embodiments, the scale comprises a stool load cell on the left side of the scale and a stool load cell on the right side of the scale. In some embodiments, the scale comprises at least one urine load cell and at least one stool load cell. For example, in some embodiments the scale comprises a urine load cell positioned on one side of the front portion of the scale (e.g., the portion proximal to where urine would be excreted from a subject using a toilet containing the scale mounted thereupon) and a stool load cell positioned on one side of the back portion of the scale (e.g., the portion proximal to where stool would be excreted from a subject using a toilet containing the scale mounted thereupon). In some embodiments, the scale comprises two urine load cells positioned on the front portion of the scale and two stool load cells positioned on the back portion of the scale, as shown in FIG. 5A. In some embodiments, the scale comprises a single urine load cell and a single stool load cell. The urine load cell and the stool cell may be positioned on the same side of the scale, or the urine load cell may be positioned on a first side and the stool load cell may be positioned on the opposite side of the scale. For example, the urine load cell and the stool load cell may each be positioned on the left side of the scale or each positioned on the right side of the scale. As another example, the urine load cell may be positioned on the left side of the scale and the stool load cell may be positioned on the right side of the scale, or vice-versa.

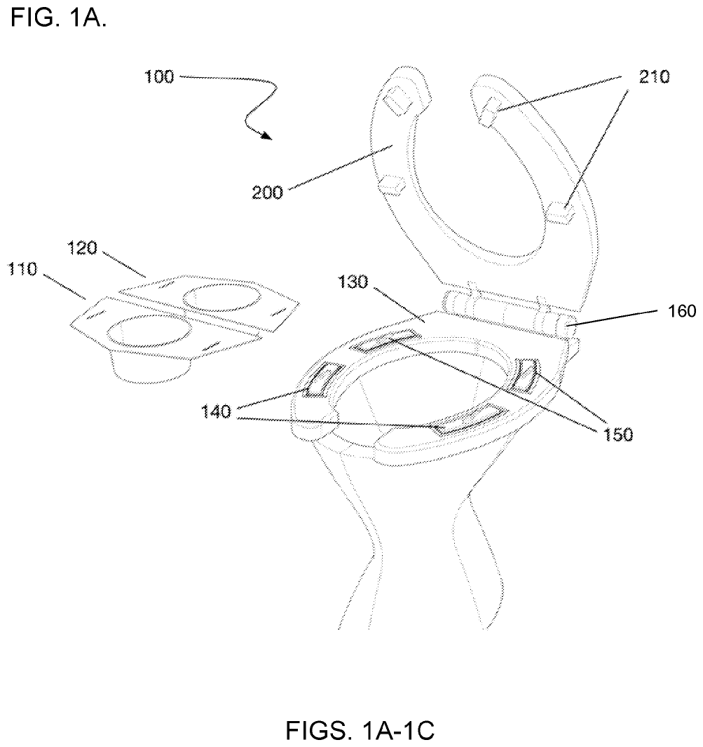

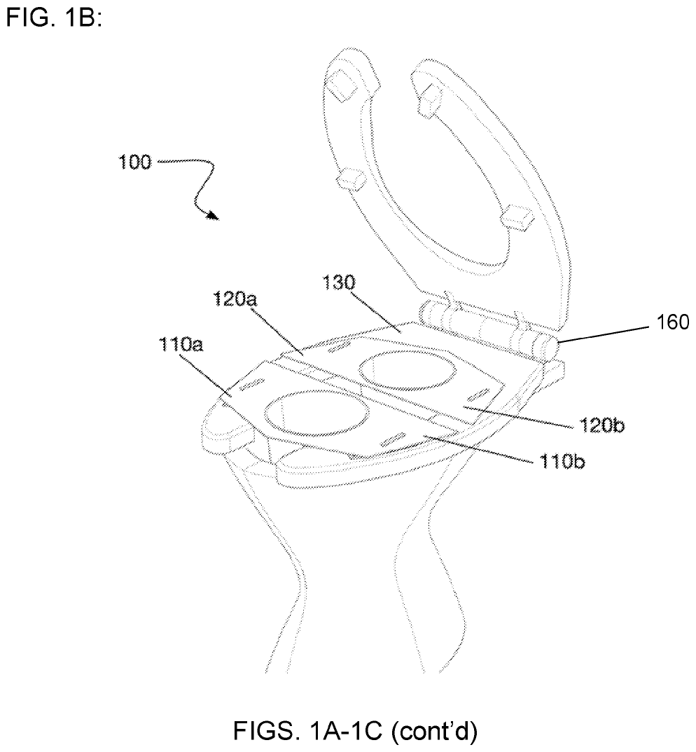

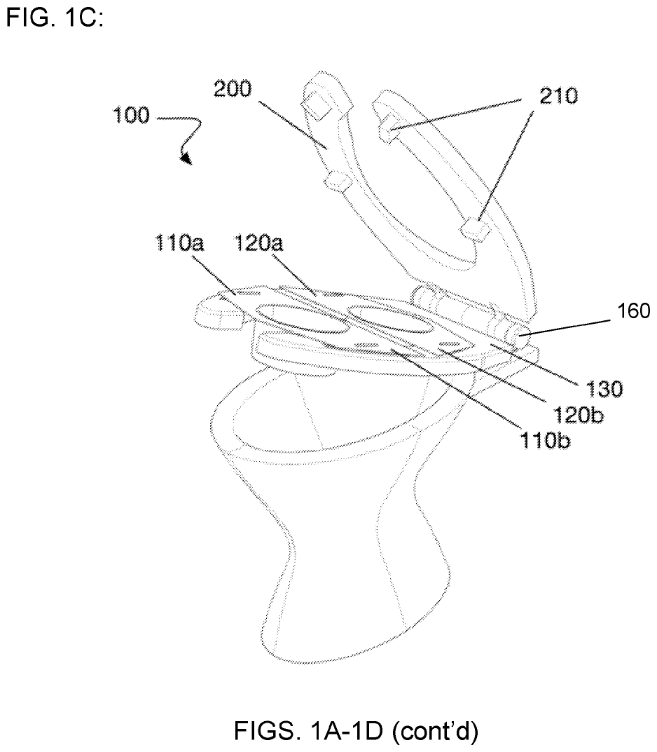

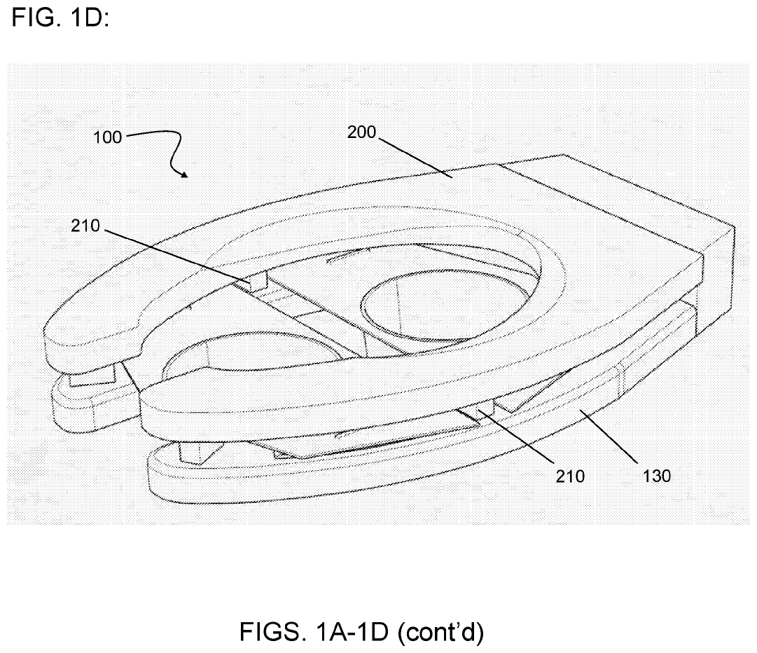

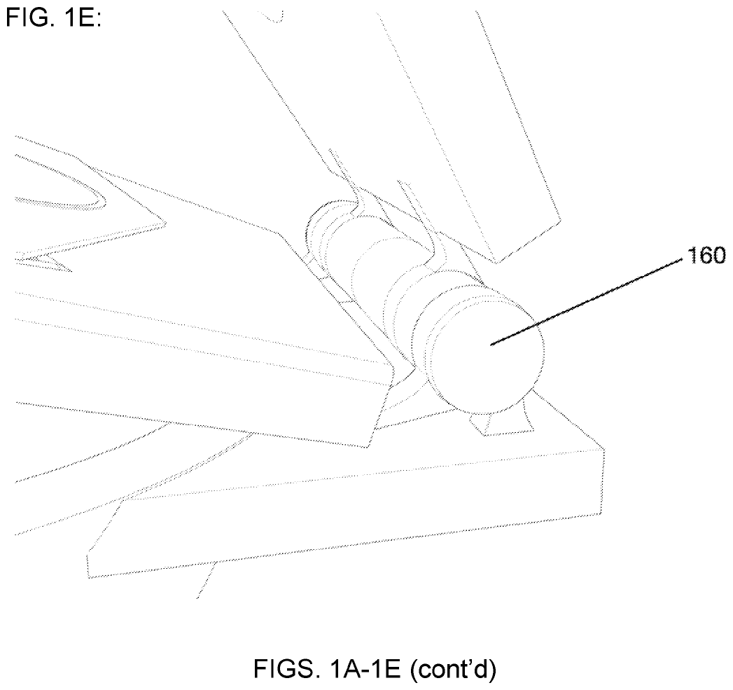

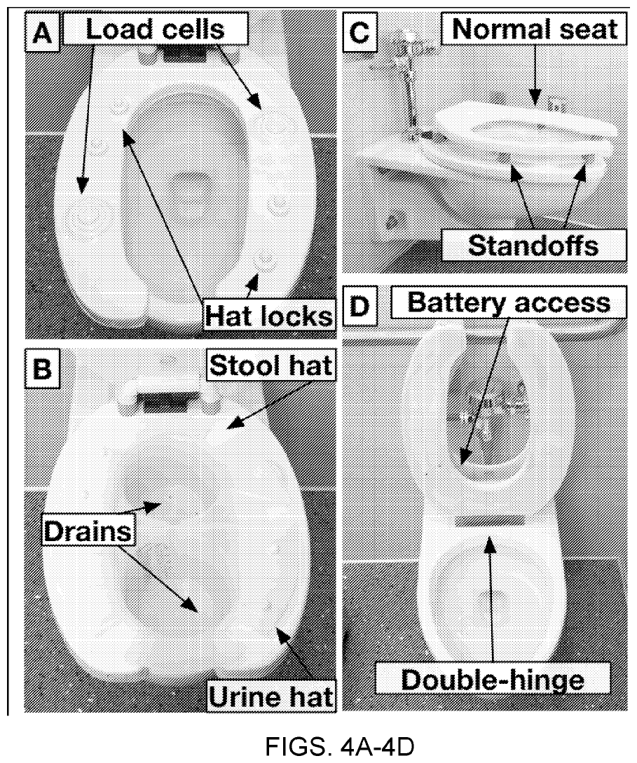

In some embodiments, the load cell is exposed, such that the load cell comes into direct contact with the overhanging region of a sample collection container. Exemplary exposed load cells are shown, for example, in FIG. 4A and FIG. 5A. In some embodiments, the loadcell is enclosed by a solid structure (e.g., plastic), as shown in FIG. 1A. In some embodiments, the load cell comprises a flexible membrane located between the solid structure and the load cell, such that weight on the solid structure is translated through the membrane and onto the load cell.

In some embodiments, the quantity measurement sensor comprises a battery powered load cell. In some embodiments, the scale further comprises a battery. In some embodiments, the battery is operably connected to the one or more load cells. In some embodiments, the battery is housed within a region of the scale containing an access panel (e.g., a door, a latch, etc.) such that the battery can be removed and replaced with a fresh battery as needed. In some embodiments, the battery is not chargeable (i.e., is disposable). In some embodiments, the battery is chargeable.

In some embodiments, the quantity measurement sensor (e.g. the scale) is configured for hinged attachment to a toilet. In some embodiments, the system further comprises at least one hinge. In some embodiments, the quantity measurement sensor further comprises a hinge that facilitates hinged attachment of the quantity measurement sensor to the toilet. For example, in some embodiments, the quantity measurement sensor scale comprises a hinge placed proximal to the back portion of the scale, thereby facilitating hinged attachment of the scale to the toilet. In some embodiments, the scale is placed on the underside of the scale at a location proximal to the back portion of the scale. Use of a hinged attachment of the quantity measurement sensor (e.g. scale) to the toilet enables the quantity measurement sensor (e.g. scale) to be lifted away from the toilet rim, thus permitting easy cleaning of various components of the toilet (e.g., the toilet base, the toilet rim, the toilet bowl) and the quantity measurement sensor (e.g., the underside of the scale, the edges of the scale, etc.).

In some embodiments, the quantity measurement sensor (e.g. the scale) further comprises a sensor locking feature in the form of one or more locking protrusions. The locking protrusions may be sized, spaced, and shaped appropriately to be used in conjunction with a suitable collection container comprising a receptacle locking feature in the form of one or more opposing holes in the overhanging region, such that the locking protrusions on the scale insert into the one or more holes of the overhanging region of the sample collection container. Use of such locking protrusions assists in securing the sample collection container in place and preventing substantial movement of the collection container during use. In some embodiments, the one or more locking protrusions exist on one side of the scale, and the one or more load cells exist on the opposite side of the scale, for each respective sample collection receptacle. Such an embodiment is shown, for example, in FIG. 1. For example, in some embodiments one or more locking protrusions for the urine receptacle exist on one side of the scale, and the urine load cell exists on the opposite side of the scale. In some embodiments, one or more locking protrusions for the stool receptacle exist on one side of the scale, and the stool load cell exists on the opposite side of the scale. As shown in FIG. 1, the locking protrusions and the load cells for the urine and stool receptacles may take opposing formations (e.g., the locking protrusions for the urine receptacle exist on the right side and the urine load cell exists on the left, whereas the locking protrusions for the stool receptacle exist on the left side and the stool load cell exists on the right, or vice-versa).

In some embodiments, the one or more locking protrusions are substantially circular in shape. In some embodiments, the one or more locking protrusions are substantially rectangular in shape. In some embodiments, the one or more locking protrusions are substantially square shaped. In some embodiments, the one or more locking protrusions extend (e.g., protrude) up from the top surface of the scale by about 1 cm to about 10 cm. For example, in some embodiments the one or more locking protrusions extend about 1 cm, about 2 cm, about 3 cm, about 4 cm, about 5 cm, about 6 cm, about 7 cm, about 8 cm, about 9 cm, or about 10 cm. In some embodiments, the one or more locking protrusions comprise two or more concentric levels to further enhance locking of the sample collection container into the protrusions. For example, in some embodiments the one or more locking protrusions comprise a first level that extends a first amount away from the scale and a second level that extends a second amount away from the scale. For example, the locking protrusions may comprise concentric circles, concentric rectangles, concentric squares, etc.

In some embodiments, the system further comprises one or more separators. The term “separator” refers to a component that generates a space between surfaces of components during use. In some embodiments, the system comprises a toilet seat comprising one or more separators positioned on a bottom surface of the toilet seat. Accordingly, in some embodiments the system comprises toilet seat comprising one or more separators positioned on the bottom surface of the toilet seat, such that the bottom surface has a different shape/outline than that of the bottom surface of an equivalent toilet seat not containing the separators. In some embodiments, the separators are positioned spatially on the bottom surface of a toilet seat to prevent the toilet seat from contacting the sample collection container or the quantity measurement sensor when the toilet seat is closed. In some embodiments, the separators are positioned spatially on the bottom surface of a toilet seat to prevent the toilet seat from contacting the sample collection container or the load cells when the toilet seat is closed. Accordingly, the separators can be used to prevent the weight of a subject sitting on the toilet seat from interfering with measurements of fecal and/or urine excretions, by preventing the weight of the subject contacting the toilet seat from transferring to the load cells of the scale. In some embodiments, the separators are placed spatially on the bottom surface of the toilet seat in areas that correspond to cutouts present on the overhanging region of the sample collection container (e.g., the urine receptacle, the stool receptacle). In some embodiments, the separators are placed spatially on the bottom surface of the toilet seat in areas of the scale where overhanging regions of the receptacle are not present and where the load cells are not present. Such areas may be referred to as “empty” areas on the scale.

In some embodiments, the toilet seat is configured for hinged attachment to the quantity measurement sensor. For example, in some embodiments the toilet seat is configured for hinged attachment to the scale. In some embodiments, the toilet seat (e.g., the toilet seat containing one or more separators) is connected to the scale by a hinge. In some embodiments, the toilet seat is configured for hinged attachment to the quantity measurement sensor (e.g. the scale), and the quantity measurement sensor (e.g. the scale) is configured for hinged attachment to the toilet. As such, in some embodiments, the toilet seat is connected to the scale by a hinge, and the scale is connected to the toilet base by a hinge. This is referred to herein as a “double hinge”. Use of a double hinge facilitates cleaning of all surfaces of each component of the system, including the toilet seat and the quantity measurement sensor (e.g. scale).

In some embodiments, the system further comprises at least one waste receptacle. In some embodiments, the at least one waste receptacle comprises an overhanging region (e.g. a support flange) that facilitates attachment of the waste receptacle to the toilet and prevents the waste receptacle from falling into the toilet bowel. In some embodiments, the system comprises a urine receptacle. In some embodiments, the system comprises a stool receptacle. In some embodiments, the at least one waste receptacle comprises a urine receptacle and a separate stool receptacle. A “urine receptacle” is a container that collects urine from a subject, wherein a “stool receptacle” is a container that collects stool from the subject. In some embodiments, the urine receptacle and the stool receptacle independently couple to the scale (i.e., the waste receptacle and the stool receptacle are separate entities. In some embodiments, the urine receptacle and the stool container are combined in a single sample collection container (i.e., the urine receptacle and the stool receptacle exist within a single entity/body.

In some embodiments, the urine receptacle (e.g., urine container) comprises a urine collection area and overhanging region (e.g. a support flange). In some embodiments, the stool receptacle (e.g., stool container) comprises a stool collection area and an overhanging region (e.g. a support flange). As describe above, in some embodiments the scale comprises a urine load cell that quantifies urine excretions from a subject. In some embodiments, the scale comprises a stool load cell that quantifies stool excretions from the subject. In some embodiments the scale comprises a urine load cell that quantifies urine excretions from a subject and a stool load cell that quantifies stool excretions from the subject. In some embodiments, a portion of the overhanging region of the urine receptacle contacts the urine load cell, thereby transferring weight present within the urine collection area of the urine receptacle to the urine load cell. In some embodiments, a portion of the overhanging region of the stool receptacle contacts the stool load cell, thereby transferring weight present within the stool collection area of the stool receptacle to the stool load cell. In some embodiments, the sample collection container or containers (e.g., the urine container, the stool container) comprise cutouts in the overhanging region. In some embodiments, the spatial location of the cutouts on the container(s) correspond to the spatial location of one of more separators on the bottom surface of the toilet seat. Accordingly, when the toilet seat is lowered the separators do not come into contact with the load cells or the sample collection container. Such an embodiment is shown, for example, in FIG. 1. In some embodiments, the size and shape of the overhanging region of the sample collection container selected such that the overhanging region of the sample collection container does not come into contact with the load cell, without requiring cutouts. Such an embodiment is shown, for example, in FIG. 4 and FIG. 5.

In some embodiments, the at least one waste receptacle comprises one or more drains in the sample collection area that permit excretions from the subject to exit the container and pass into the toilet bowl. For example, in some embodiments the urine receptacle comprises one or more drains on a bottom surface of the urine collection area, thus permitting urine to exit the container. In some embodiments the stool receptacle comprises one or more drains on a bottom surface of the stool collection area, thus permitting liquid/loose stools (e.g., diarrhea) to exit the container while solid stool remains within the container. Such a sample collection container would be particularly useful for determining the portion of a subjects fecal excretions that qualify as diarrhea opposed to solid stools. In some embodiments, the bottom surface of the collection container (e.g., the bottom surface of the urine collection area, the bottom surface of the stool collection area) is sloped toward the one or more drains.

In some embodiments, the at least one waste receptacle (e.g., urine receptacle) comprises one or more slopes to direct flow of liquid towards the one or more drains. In some embodiments, the one or more drains can be plugged (e.g., blocked), such as if a urine sample needs to be collected from the subject. In some embodiments, the at least one waste receptacle (e.g., urine receptacle) comprises one or more chamfers to direct flow of liquid towards the one or more drains.

In some embodiments, the one or more drains are sized and configured such that urine accumulates in the container before the container fully drains. In some embodiments the one or more drains are sized and configured such that the container is not void of urine between the start and end of urination, but rather becomes void of urine (e.g., empty) after urination is completed by the subject. In some embodiments, this enables urine to accumulate at a rate that allows for accurate calculation of urine volumes. The rate at which the urine (or liquid stools/diarrhea) drains out of the container can be controlled by the size, shape, and number of drains in the container. In some embodiments, the system (e.g., the one or more load cells) weighs the container continuously over time as it empties.

In some embodiments, the system further comprises a controller (e.g., a computer). In some embodiments, the controller comprises computing hardware and software, including a processor, memory, software, and/or electronic/network communications features. In some embodiments, the controller is operably connected to the one or more load cells. For example, in some embodiments the one or more load cells transmit signals to the controller (e.g., computer) for further processing by the controller. In some embodiments, the controller is contained within the scale. For example, in some embodiments the controller is embedded within the scale. In some embodiments, the controller is external to/separate from the other components of the system. For example, the one or more load cells may transmit signals wirelessly from the load cell to an external controller. In some embodiments, the controller evaluates excretions from the subject based upon information gathered and transmitted to the controller by the one or more load cells. In some embodiments, the controller calculates the mass and/or volume of excretion based on factors such as the geometry of the hat, weight change over time, emptying rate, and/or material properties of the container. Exemplary methods for calculating the mass and/or volume of excretion are shown in the accompanying examples. The cumulative data can be summarized and reported over a prescribed time period to assist in patient monitoring.

In some embodiments, the scale and the controller are configured to measure the collected waste in real time as the patient uses the toilet. For example, in some embodiments the load cells transmit signals to the controller to record a time-dependent change in weight of the collection container and calculate a mass and/or volume of waste at the end of a waste event. In other embodiments, the volume of input urine and stool is calculated by measuring liquid levels in the container over time. In some embodiments, the system calculates the liquid content of stool. This can be accomplished by, for example, calculating the initial weight of the captured stool and measuring how much it decreases over time. Whatever stool weight is “lost” as it drains provides an estimate of liquid content (e.g., diarrhea).

In some embodiments, the system further comprises one or more sensors such as cameras, ultrasound detectors, radar, emitter-detector time of flight sensors, IR, microphone, capacitance, photogate, flow sensors, etc. The sensors can be mounted on the toilet in any suitable location, such as beneath the seat or in the bowl. The waste level in the container can optionally be calculated by any other suitable method. One non-limiting example includes using a floating marker to track liquid height. Another example is to locate the outlet hole, along with an optional extension tube of a prescribed length and diameter, in a side wall of the container. The flow trajectory of the liquid leaving the container can be measured by one or more sensors of any suitable type. For example, the distance and/or angle that the flow stream travels can be captured by video as the liquid exits the container. This can be used to calculated liquid height, which can then be used to calculate a waste volume.

In some embodiments, data related to the elimination event is transmitted to and saved by the controller. The controller in turn can use the data for recording, analysis, storage, display, etc. In some embodiments, the controller transmits data to an electronic health system, such as for evaluation by a medical professional (e.g. a physician, a nurse, etc.). The term “electronic health system” is used in the broadest sense and includes a variety of suitable electronic systems, databases, records, and the like, including any system that can be used for monitoring patient treatment, safety, efficiency, and the like. In some embodiments, the electronic health system is a medical device hub or a hospital records system. For example, in some embodiments the controller is operably connected to a medical device hub and/or hospital medical record systems for real-time reporting and tracking of patient waste output and updating of patient fluid and health status. In some embodiments, the controller receives a signal from the load cells, and transmits data to external devices and/or systems. Exemplary systems that the controller can transmit data to include, for example, a flush control system, nurse monitoring stations, clinical test recorders, electronic medical records (EMR). Electronic Health Records (EHR), and the like. In some embodiments, controller is capable of analyzing or modifying the received data and can provide instructions to the external systems. Data that can be sent to electronic health systems includes, but is not limited to, subject identity, weight, height, gender, disease indications, medications being taken, and urine/stool characteristics (e.g., mass, volume, frequency, length of time, time of day), as well as any other clinically relevant data/information. In some embodiments, the controller includes an internal power supply (e.g., a battery, including a replaceable or a rechargeable battery).

The controller and processes related thereto can be implemented in hardware, software, firmware, or combinations of hardware, software and/or firmware. In some examples, the controller or processes related thereto may be implemented using a non-transitory computer readable medium storing computer executable instructions that when executed by one or more processors of a computer cause the computer to perform operations. Computer readable media suitable for implementing the control systems described in this specification include non-transitory computer-readable media, such as disk memory devices, chip memory devices, programmable logic devices, random access memory (RAM), read only memory (ROM). optical read/write memory, cache memory, magnetic read/write memory, flash memory, and application-specific integrated circuits. A computer readable medium may be located on a single device or computing separator or may be distributed across multiple devices or computing separators.

The system can optionally save energy by powering some circuitry on only when a patient is present. This can be accomplished via a sensor. Example sensors for detecting patient presence include passive infrared sensor, capacitance sensor, electrodes on the surface of the toilet seat to measure impedance, vibration sensor, accelerometer, microphone, etc. In an exemplary method, the system is configured to identify a specific patient who is sitting on the toilet and links the urine volume measurement to the patient unique identifier when transmitting the data from the toilet system to an external computing device. Example sensor choices for uniquely identifying the patient include determining the location of person as given by a location tracking system and locating them within a geofenced area around the toilet, or an RFID tag that the patient is wearing and uniquely identifies the patient.

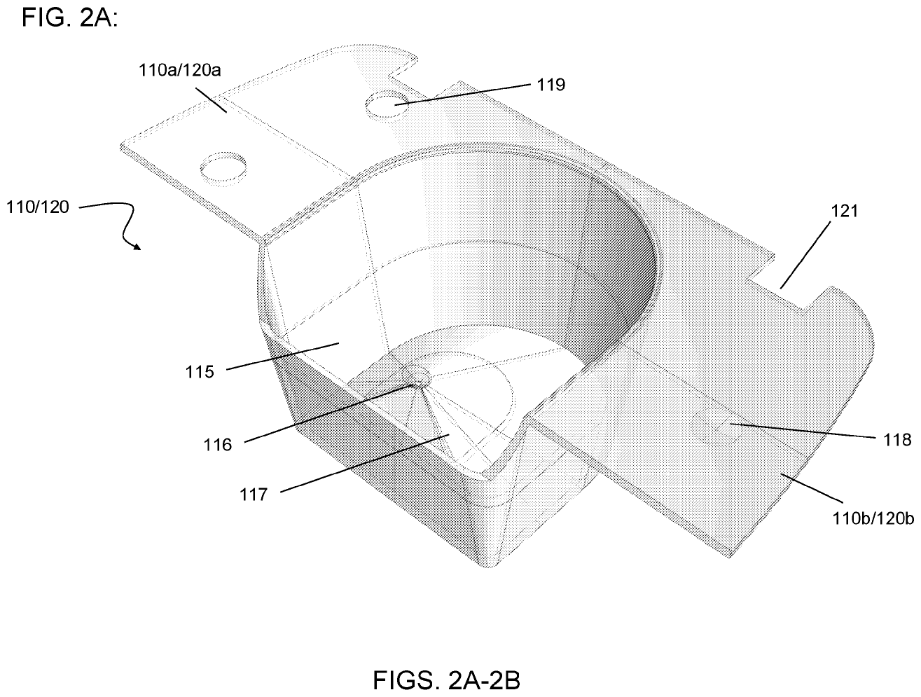

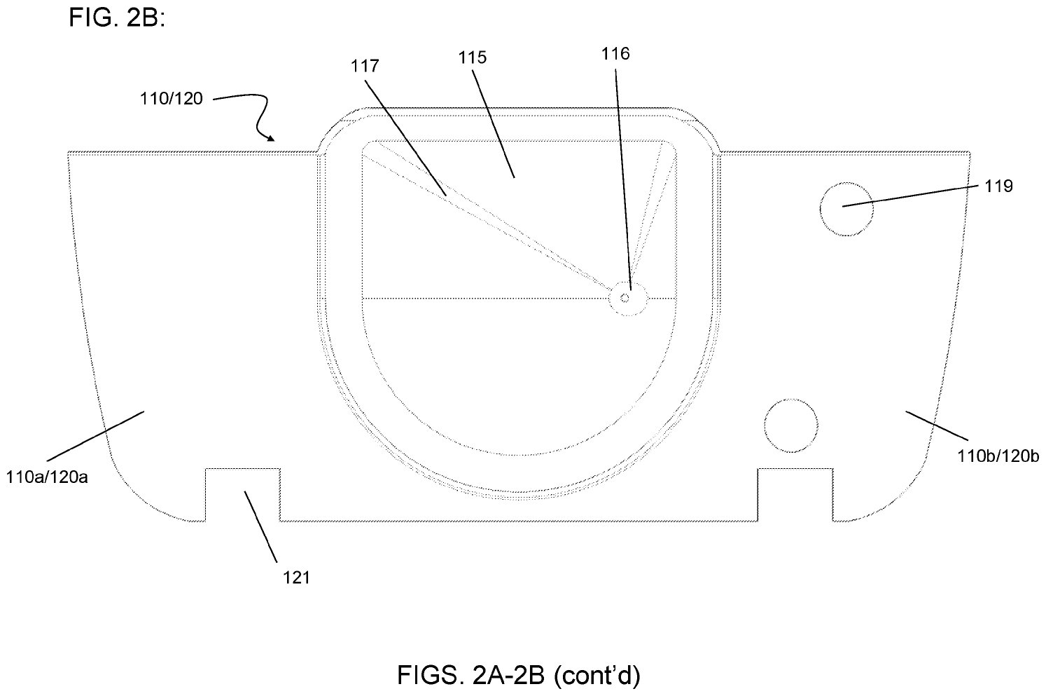

Referring in more detail to exemplary embodiments, FIGS. 1A-1E show representative illustrations of an exemplary system for evaluating excretions from a subject, according to one embodiment of the present disclosure. FIG. 1A is an exemplary illustration showing the system (100) with the toilet seat (200) raised and without the sample collection containers (e.g., urine receptacle (110), stool receptacle (120)) placed upon the scale (130). The toilet seat (200) is attached to toilet by a hinge (160), and the scale (130) is attached to the toilet by the hinge (160). Such a hinge is referred to herein as a “double-hinge”. The scale comprises two urine load cells (140) and two stool load cells (150). The urine load cells are located towards the front of the seat (e.g., proximal to the area where urine would be released from a subject) and the stool load cells are located towards the rear of the seat (e.g., proximal to the area where stool would be released from a subject). The system (100) comprises a toilet seat (200), shown as raised in the figure. The underside surface of the toilet seat comprises four separators (210). The separators are placed positionally such that they will not come into contact with the load cells when the seat is lowered. FIG. 1B is an exemplary illustration showing placement of the sample collection containers (110 and 120) upon the scale (130). The sample collection containers comprise overhanging regions and a sample collection area. The overhanging regions (110a and 110b; 120a and 120b) are shaped such that a portion of the overhanging region will come into contact with the load cells (140 and 150). The urine receptacle comprises overhanging regions that come into contact with the urine load cells, and the stool receptacle comprises overhanging regions that come into contact with the stool load cells. The overhanging regions are additionally configured to have cutouts (see FIGS. 2A-2B) such that the separators (210) on the underside surface of the toilet seat will not contact the overhanging regions when the seat (200) is lowered, but will rather fit inside the cutouts and contact the scale directly. FIG. 1C is an exemplary illustration showing a view of the system (100) when the seat (200) is partially closed and the scale (130) is partially lifted, exemplifying how the double-hinge (160) facilitates movement of both components of the system. FIG. 1D is an exemplary illustration showing a view of the system (100) when the seat (200) is closed, demonstrating the proper positioning of the separators (210) on the underside surface of the toilet seat such that the load cells and the overhanging regions of the sample collection container are not contacted by the separators when lowered. FIG. 1E shows a close-up view of the double hinge (160).

FIGS. 2A-2B show representative illustrations of an exemplary sample collection container for use in the systems and methods provided herein, according to one embodiment of the present disclosure. FIG. 2A shows an exemplary illustration of the collection container (110/120). The container comprises an overhanging region and a sample collection area (115). The bottom surface of the sample collection area contains a drain (116). Multiple drains may be used. The container may also comprise slopes (117) which direct the flow or liquid (e.g., urine) towards the drain (116). The overhanging region may comprise depressions (118) which facilitate contact between the sample collection container and the scale (130). The overhanging region can additionally comprise one or more holes (119) which facilitate securement of the sample collection container to the scale. The sample collection container comprises cutouts (121). The one or more separators can be placed positionally on the underside surface of the toilet seat such that they will lower into the cutouts (121) of the sample collection container. FIG. 2B shows top view of the exemplary sample collection container.

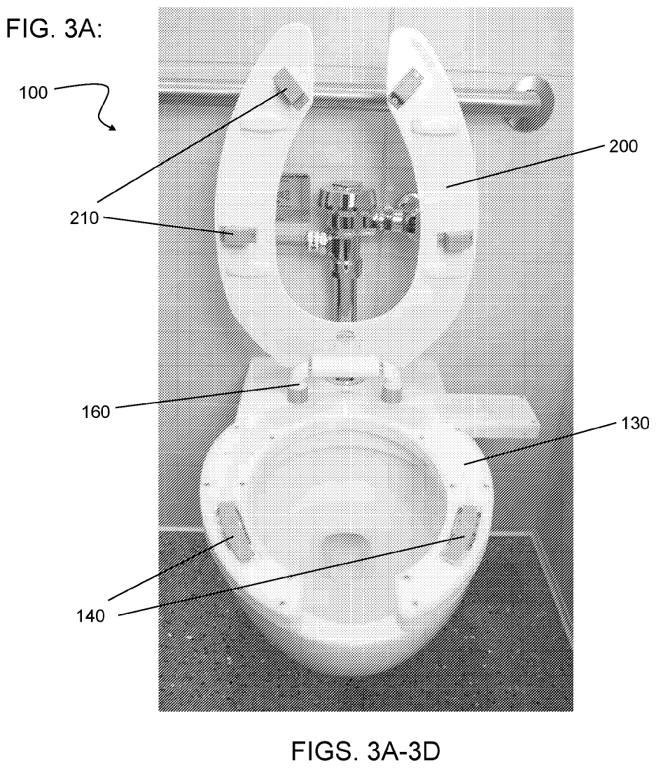

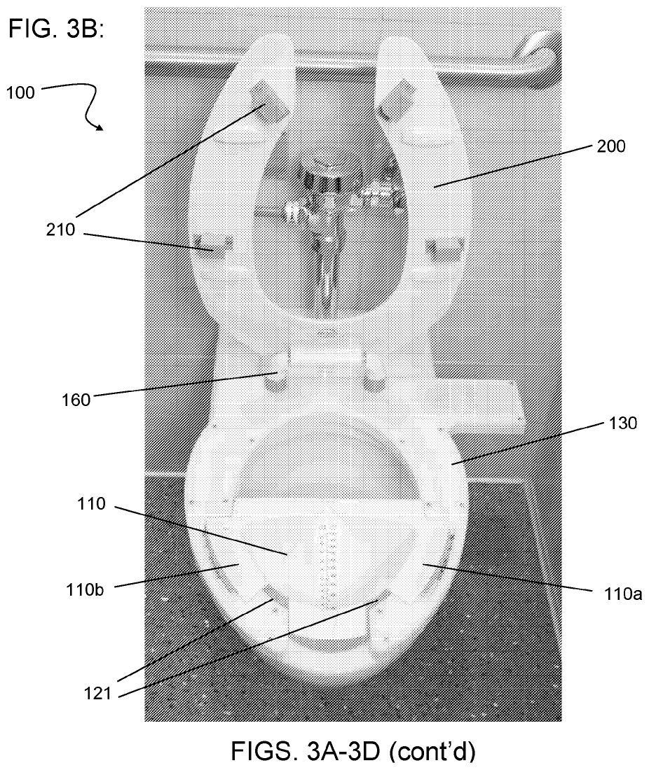

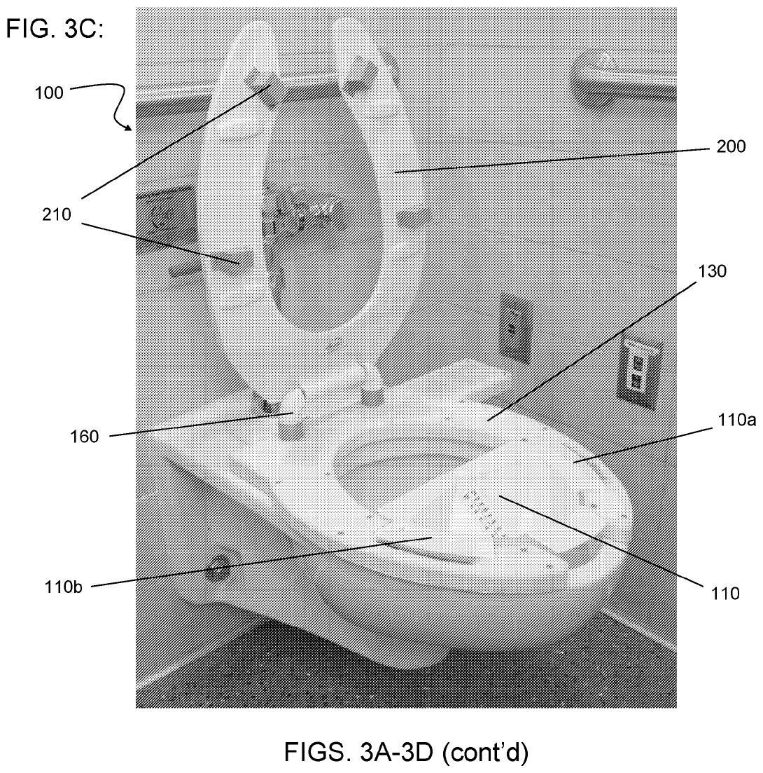

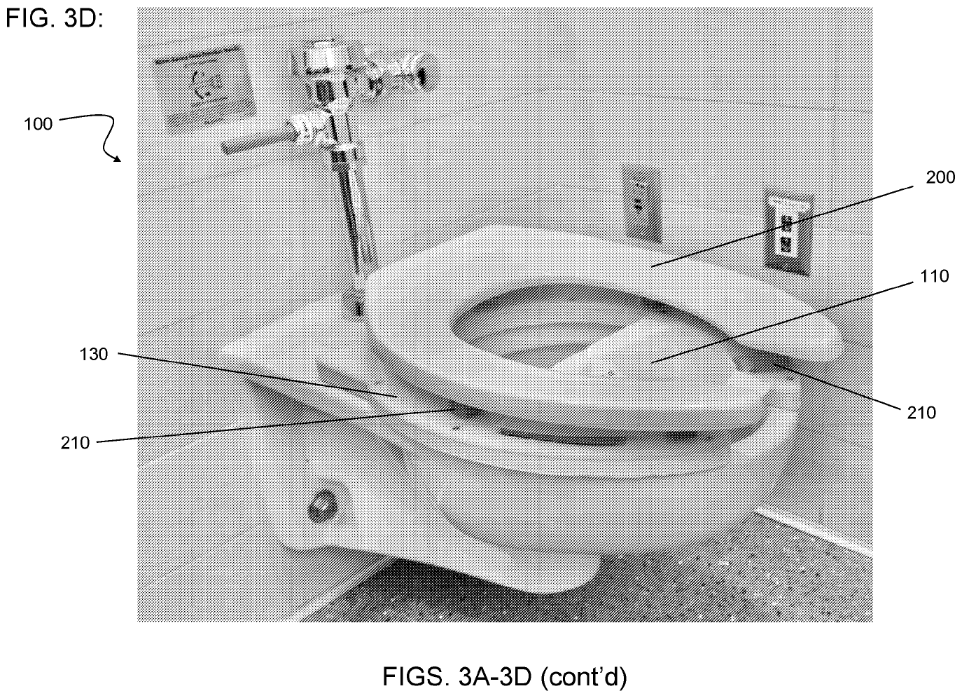

FIGS. 3A-3D show representative photographs of an exemplary system for evaluating urine excretions from a subject, according to one embodiment of the present disclosure, located in a hospital bathroom. FIG. 3A shows a photograph of an exemplary system (100), absent a sample collection container. The system comprises a scale (130), housed above the toilet rim and below the toilet seat (200). The scale contains two urine load cells (140) for measuring urine. The system comprises a toilet seat (200), the underside surface of which is equipped with four separators (210). The top surface of the scale (130) comprises a single hinge (170), which attaches to the underside of the toilet seat (130) and permits the seat to be lifted away from the scale. FIG. 3B shows a view of the system containing a sample collection container (e.g., urine collection container). The urine collection container comprises overhanging regions (110a, 110b) and cutouts (121), along with a urine collection area. A portion of the overhanging regions come into contact with the load cells, whereas the cutouts do not. FIG. 3C shows a side view of the system shown in FIG. 3B. FIG. 3D shows a view of the system with the toilet seat lowered. The separators (210) on the toilet seat (200) come into contact with “empty” spaces on the scale (130) (e.g., cutouts of the urine collection container and/or locations on the scale that are not the load cells), thus preventing weight from being transferred undesirably from the toilet seat to the load cells or to the sample collection container.

FIGS. 4A-4D show photographs of an exemplary system for evaluating excretions from a subject in accordance with one embodiment of the present disclosure. FIG. 4A shows a photograph of an exemplary scale in accordance with one embodiment of the present disclosure. The scale comprises two load cells, which weigh waste receptacles. In this embodiment, the scale comprises a urine load cell in the front of the scale and a stool load cell in the back of the scale. Locking protrusions, referred to FIG. 4A as “hat locks”, fit into equivalent holes in the overhanging regions of the waste receptacles and lock them into place. Waste receptacles resting on locking protrusions have the physical properties of a beam torqueing around a pivot point, as such weight can be measured using a load cell on only own side of the receptacle, simplifying the design and reducing battery requirements. FIG. 4B shows a photograph of an exemplary system including waste receptacles. In this embodiment, the system comprises a rear waste receptacle (i.e., a stool receptacle) that catches and weighs stool (referred to in the figure as as a “stool hat”) and a forward waste receptacle (i.e. a urine receptacle) used for urine (referred to as a “urine hat”). Drains in the bottom of the waste receptacles allow liquid to drain out. FIG. 4C is a photograph showing the system including a conventional, off-the-shelf toilet seat for patient comfort. The toilet seat comprises separators, referred to in the figure as “standoffs”, to prevent the toilet seat from directly contacting the hats or load cells and affecting weight measurements. FIG. 4D is a photograph showing a double-hinge design, which allows the scale to lift away from the toilet rim for cleaning. Waterproof battery access is also concealed beneath device.

Embodiments of the present disclosure also include a method of evaluating excretions from a subject using the any of the systems described above. In accordance with these embodiments, the method includes measuring/collecting/obtaining a signal from at least one load cell. In some embodiments, the method includes detecting a subject's presence, initiating data collection using the controller, and collecting signal from the at least one load cell. In some embodiments, the method further comprises analyzing the signal from the load cell using the controller, which can calculate the weight and/or volume of excretions (e.g. urine, stool) from a subject and/or can evaluate liquid vs. loose stool excretions from the subject. In some embodiments, the method further comprises transmitting data from the controller to an electronic health system.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

### Example 1

Provided herein is a cost-effective system for automatic evaluation of excretions from a subject. The system provided herein can evaluate urine excretions from a subject (e.g., measure the weight and/or volume of urine in an automated fashion, without the need for manual determination or calculation of output) and can differentiate between liquid stool (e.g., diarrhea) and solid stool. An exemplary system is shown in FIG. 4. As shown in FIG. 4, the system can be used in conjunction with a standard toilet, without modification to components of the toilet (e.g., the base, the bowl, the rim) or the plumbing. The system comprises a scale that attaches to the toilet above the towel rim and below the toilet seat. As shown in FIG. 4A, the scale contains load cells and locking protrusions, referred to in the figure as “hat locks”. As shown in FIG. 4B, the system comprises a urine collection container and a stool collection container, referred to in the figure as a “urine hat” and a “stool hat”, respectively. The urine collection container (e.g., the urine receptacle) and the stool collection container (e.g., the stool receptacle) can be separate container, or can exist within a single sample collection container. As shown in FIG. 4B, the collection containers contain drains to allow liquid to exit the container. The collection containers further comprise cutouts and holes in the overhanging region. The locking protrusions of the scale insert into the holes of the overhanging regions of the collection containers, thereby securing the container to the scale. As shown in FIG. 4C, the system further comprises a standard toilet seat equipped with one or more separators, referred to in the figure as “standoffs”. The separators are placed spatially long the bottom surface of the toilet seat to prevent force from the toilet seat from transferring to the scale. Accordingly, the separators are placed spatially in locations corresponding to the locations of the cutouts in the sample collection containers, such that when the toilet seat is lowered, the separators do not come into contact with the container or the load cell. As shown in FIG. 4D, the system comprises a double-hinge, namely one hinge connecting the scale to the toilet rim and another hinge connecting the toilet seat to the scale. This hinging mechanism can also be seen in FIG. 4C.

The system analyzes load cell data over time to detect urination and defecation, as well as to quantify output and consistency (FIG. 5A). Using a set of ordinary differential equations based on Torricelli's law, a model was derived and solved that allows for calculation urine volumes using measurements of how urine drains from hats over time (FIG. 5B). The device calculates urine volume using the specific gravity of urine (1.002 g/ml; this value only varies by 3% across a range of hydration states). Coefficients (C, C_{d}; FIG. 5C) allow the mathematical model to be fitted with hats featuring different geometries and drain diameters.

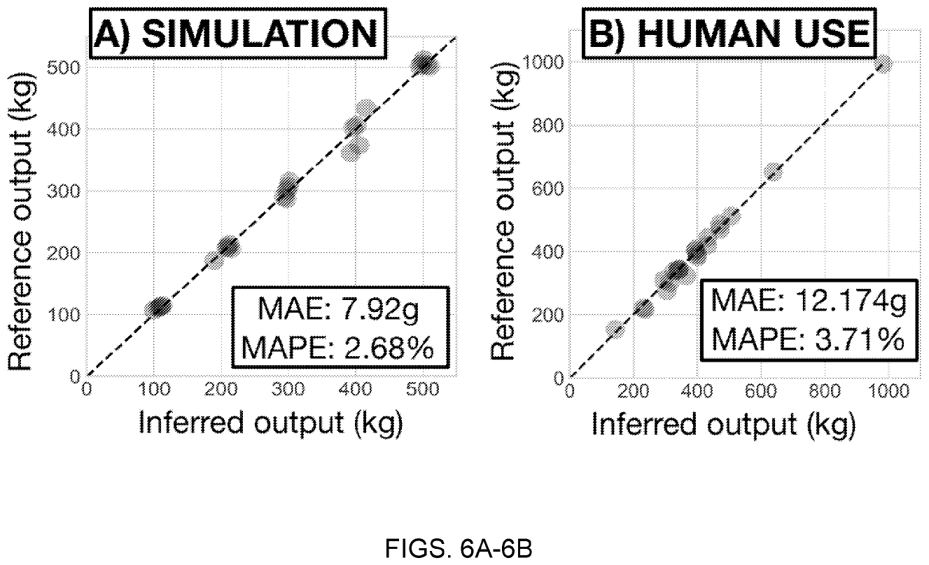

Accuracy: Both synthetic and real-world tests of the system indicate accuracy on the order of grams. In one test, known volumes of water (100 to 500 mL) were poured at a rate that resembled urination (˜10 ml/s) into the urine-catching component of the device (n=25 trials). Comparison of the resulting device measurements to the true water volumes revealed a mean absolute error of just 7.92 g or 2.68% per urination event (FIG. 6A). Based on the strength of these benchtop tests, a cohort of healthy volunteers was recruited to test the device. Volunteers varied by physiology (6 men, 6 women; 5′4″-6′1″ height; 110-265 pounds: 24-54 years old). The devices were tested more than 250 times, with high accuracy achieved. In an exemplary test, participants were asked to urinate into the device (n=43 total uses). Calculated urine outputs were compared to the difference in participant weight before and after device use, which was measured by a reference scale in the bathroom. Half of the uses were used to “train” an ODE-based algorithm and infer the coefficients in Eq. 4 (FIG. 5C) that account for hat geometry and drain diameter. The resulting model was applied to the held-out “test” set of remaining samples, which confirmed that the system and method is highly accurate at calculating urine output with a mean absolute error of 12.174 g or 3.71% of each urination event (FIG. 6B).

Hospital-optimized features: The system fits onto standard toilet seat mounts and can be installed in minutes without plumbing modification in bathrooms, including hospital or care facility bathrooms. A conventional, “off-the-shelf” molded toilet seat can be used, equipped with separators, and all outer edges of the components of the system are beveled to minimize injury if patients fall on the system (FIG. 4C). The system also lacks exterior wires or floor-mounted components to avoid tripping hazards. The system includes a “slow-close” mechanism in its hinges, which gently lowers the scale and toilet seat during closing to prevent jarring the seat's electrical components or pinching patient fingers. The system was tested in accordance with best practices for hospital bathroom disinfection. All crevices in which liquid could pool were eliminated, and all surfaces are finished with a smooth epoxy. The system also double-hinged (FIG. 4D) so that its underside can be lifted and cleaned.

Computing, networking, and power requirements: Load cells are driven by an Atmel microcontroller running an Arduino operating system, although any suitable controller and operating system can be used. In some embodiments, a Bluetooth module transcripts data to a separate station, such as a physician station or a nursing station to monitor patient data. The station (e.g., nurses station, physicians station such as a computer or a tablet) displays patient data in real-time to healthcare staff. This computing structure is also designed to be low-power and minimize shock risks in patient bathrooms.

1. An excretion collection and evaluation system comprising:
a) at least one waste receptacle including a waste collection area; and
b) a sensor configured to be coupled to a toilet, selectively coupled to the at least one waste receptable, and to generate a signal indicative of a quantity of waste deposited in the waste collection area.

2. The excretion collection and evaluation system of claim 1, wherein the sensor includes at least one load cell that interfaces with the at least one waste receptacle.

3. The excretion collection and evaluation system of claim 1, wherein the at least one waste receptacle includes an overhanging region selectively coupled to the sensor.

4. The excretion collection and evaluation system of claim 3, wherein the overhanging region includes a receptacle locking feature configured to constrain movement of the at least one waste receptacle relative to the sensor.

5. The excretion collection and evaluation system of claim 3, wherein the sensor includes a sensor locking feature configured to engage the at least one waste receptacle to constrain movement of the at least one waste receptacle relative to the sensor.

6. The excretion collection and evaluation system of claim 5, wherein the receptacle locking feature includes an aperture, and the sensor locking feature includes a protrusion sized to engage the aperture.

7. The excretion collection and evaluation system of claim 1, wherein the at least one waste receptacle includes a urine receptacle and a stool receptacle, wherein the urine receptacle and the stool receptacle are independently coupled to the sensor.

8. The excretion collection and evaluation system of claim 1, wherein the at least one waste receptacle includes a urine receptacle and a stool receptacle, wherein the urine receptacle and the stool receptacle are combined in a single sample collection container.

9. The excretion collection and evaluation system of claim 1, wherein the at least one waste receptacle includes a urine receptacle.

10. The excretion collection and evaluation system of claim 1, wherein the at least one waste receptacle includes a stool receptacle.

11. The excretion collection and evaluation system of claim 1, wherein the at least one waste receptacle includes a drain.

12. The excretion collection and evaluation system of claim 11, wherein the at least one waste receptacle defines slopes to direct a flow of fluid towards the drain.

13. The excretion collection and evaluation system of claim 1, further comprising at least one separator configured to inhibit contact between a toilet seat, the sensor, or the at least one waste receptacle, wherein the at least one waste receptacle includes a cutout sized to receive the separator, and wherein the separator is secured to the toilet seat.

14. The excretion collection and evaluation system of claim 1, further comprising a controller that receives the signal from the sensor and evaluates a weight or volume of excretions based on the signal.

15. The excretion collection and evaluation system of claim 14, wherein the controller is configured to transmit data to an electronic medical station.

16. An excretion collection and evaluation system comprising:
a) a toilet seat configured to be hingedly coupled to a toilet and including a separator extending from a bottom surface of the toilet seat; and
b) a quantity measurement sensor coupled to the toilet seat and positioned so that the separator inhibits contact between the toilet seat and the quantity measurement sensor.

17. The excretion collection and evaluation system of claim 16, wherein the quantity measurement sensor includes a battery powered load cell.

18. The excretion collection and evaluation system of claim 16, wherein the quantity measurement sensor is configured to be directly coupled with the toilet and the toilet seat includes a toilet seat hinge configured to couple the toilet seat to the quantity measurement sensor.

19. A waste receptacle for use with an excretion collection and evaluation system, the waste receptacle comprising:
a. a waste collection area;
b. a support flange extending from the waste collection area and including a receptacle locking feature, the support flange configured to:
i. suspend the waste collection area within a toilet bowl,
ii. constrain movement of the waste receptacle relative to the excretion collection and evaluation system, and
iii. engage a quantity measurement sensor of the excretion collection and evaluation system; and
c. a drain positioned within the waste collection area configured to allow excretions to exit the waste collection area during use.

20. The waste receptacle of claim 19, wherein the waste collection area includes a urine receptacle and a separate stool receptacle.
